(19) United States
(12) Patent Application Publication
(10) Pub. No.: US 2025/0145659 A1
(43) Pub. Date: May 8, 2025

(54) SYNTHESIS OF CHOLESTEROL FORM BISNORALCOHOL

(71) Applicant: FERMENTA BIOTECH LIMITED, Thane West (IN)

(72) Inventors: Anupama DATLA, Mumbai (IN); Prashant NAGRE, Thane West (IN); Jagdish TAMORE, Thane West (IN); Sachin Govind WADHAVANE, Navi Mumbai (IN); Amol SHIRSATH, Ambernath EAST (IN); Gajanan Subhash GAONKAR, Thane (IN)

(21) Appl. No.: 18/837,486

(22) PCT Filed: Feb. 11, 2023
(86) PCT No.: PCT/IN2023/050138

§ 371 Date: Aug. 9, 2024

(30) Foreign Application Priority Data
Feb. 11, 2022 (IN) 202221007425

(51) Int. Cl. C07J 9/00

(52) U.S. Cl. CPC C07J 9/00

(57) ABSTRACT

The present invention discloses a short, cost effective process for preparation of cholesterol from bisnoralcohol (BA).

### FIELD OF THE INVENTION

The present invention relates to a short, cost effective process for preparation of cholesterol from bisnoralcohol (BA).

### BACKGROUND OF THE INVENTION

Cholesterol is a sterol a type of lipid. Cholesterol is biosynthesized by all animal cells and is an essential structural component of animal cell membranes. When chemically isolated, it is a yellowish crystalline solid.

Cholesterol also serves as a precursor for the biosynthesis of steroid hormones, bile acid and Vitamin D3- or Cholecalciferol, Alfacalcidiol or Calcitriol. Cholesterol is the principal sterol synthesized by all animals.

Commercially available cholesterols (24 samples) have a purity in the range of 90.7 to 96.3% as determined by assay through GLC. In recent years, researches have been actively conducted on drug delivery system in the medical field. Currently investigations are being made into, as one of the system means, liposomes having a medicament covered with a membrane composed of phospholipid and cholesterol. In this connection, the purity of cholesterol useful as the starting material has come into question. And now there is an increased need for cholesterol of higher purity.

The processes in the art are lengthy and cumbersome. Traditionally cholesterol is extracted from animal source mainly wool grease or Fish oil residue. The present inventors envisaged that there is a scope to synthesize cholesterol from bisnoralcohol in fewer steps. This remains the objective of the invention.

### SUMMARY OF THE INVENTION

Accordingly, the present invention discloses a short, cost effective process for the preparation of cholesterol from bisnoralcohol comprising;
1. Tosylating Bisnoralcohol to obtain bisnoralcohol tosylate (2);
2. Converting the compound (2) to 3-Ketal Bisnoralcohol Tosylate (3);
3. Treating the compound (3) with Grignard reagent to obtain 3-Ketal Cholestenone (4);
4. Converting compound (4) to give Cholest-4-ene-3-none (5);
5. Acetylating compound (5) to give 3-Acetoxy-Cholest-3,5-diene (6); and
6. Reducing the compound (6) to obtain cholesterol.

### DETAILED DESCRIPTION OF THE INVENTION

The invention will now be described in detail in connection with certain preferred and optional embodiments, so that various aspects thereof may be more fully understood and appreciated.

In an embodiment, the present invention relates to a short, cost effective process for the preparation of cholesterol from bisnoralcohol.

Accordingly, Step 1 of the process comprises dissolving Bisnoralcohol (1) in the solvent and treating with p-Toluene Sulphonyl Chloride (1.91-3.11 mole equivalent) preferably 2.08 mole equivalents in presence of pyridine (0.41-2.093 mole equivalent) preferably 0.41 mole equivalent at 10-40 degree preferably at 30-40 degree for 8-16 hrs preferably 12 hrs to synthesise bisnoralcohol tosylate (2).

The process is carried out in presence of base such as pyridine, triethyl amine, imidazole, Diisopropyl ethyl amine and in the solvent selected from halogenated hydrocarbons, toluene, esters and the like.

Step 2: The bisnoralcohol tosylate (2) in treated with Ethylene Glycol (7.82 to 15.63 mole equivalence) preferably 15.63 mole equivalence in presence of alkyl ester of orthoformic acid (2.28 to 9.14 mole equivalence) preferably 2.28 mole equivalence at 25 to 120 degree, preferably at 25-32 degree to synthesise 3-Ketal Bisnoralcohol Tosylate (3). The ketal formation may be carried out in presence of Propane1,3 diol. For the quenching of water molecule alkyl ester of orthoformic acid is used such as trimethyl orthoformate, triethyl orthoformate and the like. For the reaction progress in forward direction weak acids are used such as Para toluene sulphonic acid monohydrate.

Step 3: The 3-Ketal Bisnoralcohol Tosylate (3) is treated with Isopentyl Magnesium Bromide (2 to 8 mole equivalence) preferably in 4 mole equivalence in THF at 0-25 degree preferably at 15 degree with cuprous bromide catalyst 0.025 mole equivalence to yield 3-Ketal Cholestenone (4). The coupling reaction with Grignard reagent is carried out in presence of cuprous bromide, dimethyl sulphide in the solvent such as THF, diethyl ether, 3-THF.

Step 4: The 3-Ketal Cholestenone (4) is treated with p-Toluene sulphonic acid 0.58 mole equivalence and water at room temperature to yield Cholest-4-ene-3-none (5). The deprotection of cyclic ketals is carried out using acid and water. the acids are selected from organic or inorganic acid.

Step5: Cholest-4-ene-3-none (5) is treated with acylating agents to form enol esters, acetylating agents used as acetic anhydride (11.34 to 22.68 mole equivalence) preferably 11.34 mole equivalence or Isopropenyl acetate (3.99 to 7.72 mole equivalence) preferably 3.99 mole equivalence in presence of acid catalyst at 100-110 degree to yield 3-Acetoxy-Cholest-3,5-diene (6). The acetylation produces acetyl ester of alpha beta unsaturated ketone.

Step 6: Step 5 enol esters (6) are treated with in situ-generation of calcium borohydride (reacting sodium borohydride 2.44 mole equivalence with calcium chloride anhydrous mole equivalents) 0-25 degree for 12-36 hrs to yield cholesterol as major product and epicholesterol as side product. The alkali metal metal borohydrides are selected from sodium borohydride, calcium borohydride, lithium borohydride and the solvent is selected from lower alcohols such as ethanol, methanol, Isopropanol or t-butanol alone or mixtures thereof. The process is depicted in the scheme below:

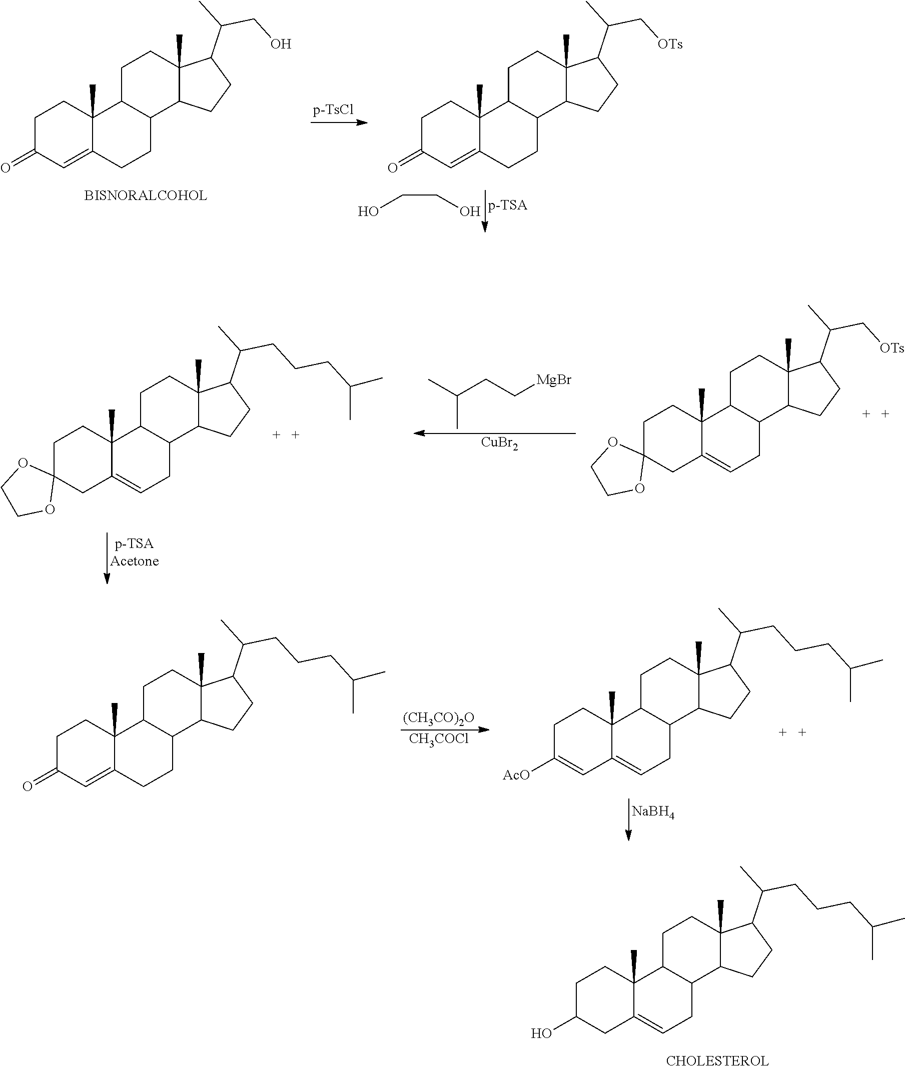

The invention will now be described in the following specific examples, however, it is being understood that the particulars shown are solely for purpose of illustrative discussion of preferred embodiments of the invention.

### EXAMPLE

### Step 1: Synthesis of BISNOR TOSYLATE (2)

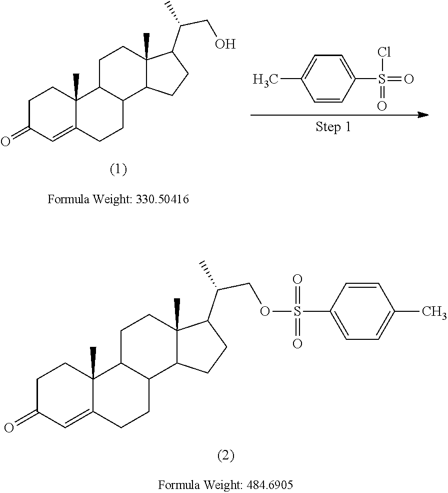 [SMILES: C[C@H](CO)C1CCC2C3CCC4=CC(=O)CC[C@]4(C)C3CC[C@@]21C ; Cc1ccc(S(=O)(=O)Cl)cc1 ; Cc1ccc(S(=O)(=O)OC[C@@H](C)C2CCC3C4CCC5=CC(=O)CC[C@]5(C)C4CC[C@@]32C)cc1]

1. 100 gms (302 millimole) of bisnoralcohol was suspended in 500 gm (632.1 millimole) of pyridine and 500 ml Dichloromethane along with 120 gms (629 millimole) of para toluene sulphonyl chloride at 25-35° C. and the reaction mass stirred for 12-18 hours till the TLC analysis indicates the absence of starting material wash with 500 ml 1:1 HCL solution followed by 500 ml water and finally with 500 ml of 8% sodium bicarbonate solution dichloromethane layer evaporated to get residue. 134 gm, HPLC purity 92%, yield 90%.
2. 100 gms (302millimole) of bisnoralcohol was suspended in 100 gm (126.4 millimole) of pyridine and 500 ml Dichloromethane along with 120 gms (62.9 millimole) of para toluene sulphonyl chloride at 25-35° C. and the reaction mass stirred for 12-18 hours till the TLC analysis indicates the absence of starting material wash with 500 ml 1:1 HCL solution followed by 500 ml water and finally with. 500 ml of 8% sodium bicarbonate solution dichloromethane layer evaporated to get residue. 138 gm HPLC purity 91% yield 87%.
3. 1 gms (3.02 millimole) of Bisnoralcohol was suspended in 5 gm (63.21 millimole) of pyridine and 50 ml chloroform along with 1.8 gms (9.4 millimole) of para toluene sulphonyl chloride at 25-35°° C. and the reaction mass stirred for 12-18 hours till the TLC analysis indicates the absence of starting material wash with 50 ml 1:1 HCL solution followed by 50 ml water and finally with. 50 ml of 8% sodium bicarbonate solution dichloromethane layer evaporated to get residue. 1.4 gm yield 95%, crystalises with methanol yield 1.0 gm compound, HPLC purity 96%
4. 1 gms (3.02 millimole) of bisnoralcohol was suspended in 5 gm (63.21 millimole) of pyridine along with 1.5 gms (7.88 millimole) of para toluene sulphonyl chloride at 25-35° C. and the reaction mass stirred for 12-18 hours till the TLC analysis indicates the absence of starting material wash with 50 ml 1:1 HCL solution followed by 50 ml water and finally with. 50 ml of 8% sodium bicarbonate solution dichloromethane layer evaporated to get residue. 1.5 gm yield 95%, crystalises with methanol yield 1.1 gm compound HPLC purity 92%
5. 1 gms (3.02 millimole) of bisnoralcohol was suspended in 5 gm (73.44 millimole) of Imidazole and 50 ml Dichloromethane along with 1.1 gms (5.77 millimole) of para toluene sulphonyl chloride at 25-35° C. and the reaction mass stirred for 12-18 hours till the TLC analysis indicates the absence of starting material wash with 50 ml 1:1 HCL solution followed by 50 ml water and finally with. 50 ml of 8% sodium bicarbonate solution dichloromethane layer evaporated to get residue. 1.4 gm, crystallizes with methanol yield 1.0 gm compound HPLC purity 93%.

### STEP 2: Synthesis of 3-Ketal BisnoralcoholTosylate (3).

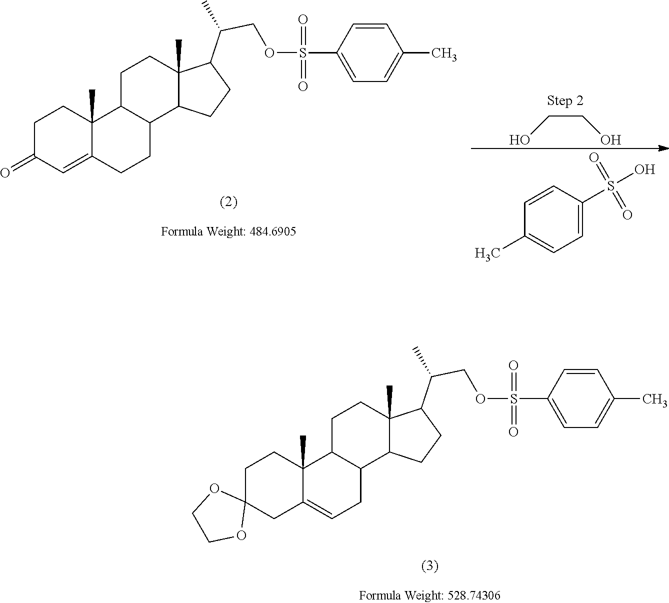

1) 100 gms (206.31 millimole) of I was suspended 500 ml Dichloromethane along with 200 gm (322.2 millimole) of Ethylene Glycol 25-35° C. and charged 200 gm (188.4 millimole) of trimethyl orthoformate. the reaction mass stirred for 12-18 hours till the TLC analysis indicates the absence of starting material. Add triethylamine to adjust pH alkaline followed by 100 ml water addition and finally with. 100 ml of 10% sodium chloride solution dichloromethane layer evaporated to get residue. 106 gm HPLC purity 87% yield 95%
2) 100 gms (206.31 millimole) of I was suspended 500 ml Toluene along with 200 ml (322 millimole) of Ethylene Glycol 25-35° C. and charged 0.1 gm of para toluene sulphonic acid, the reaction mass stirred for 18-24 hours at reflux with Dean stark apparatus till the TLC analysis indicates the absence of starting material. add triethylamine to adjust pH alkaline followed by 10 ml water addition and finally with. 100 ml of 10% sodium chloride solution dichloromethane layer evaporated to get residue. 95 gm HPLC purity 90%, yield 92%
3) 1 gms (2.06 millimole) of I was suspended 20 ml Dichloromethane along with 2 gm (32.2 millimole) of Ethylene Glycol at reflux temperature. and charged 2 gm (18.84 millimole) of trimethyl orthoformate. the reaction mass stirred for 6-12 hours till the TLC analysis indicates the absence of starting material. Add triethylamine to adjust pH alkaline followed by 10 ml water addition and finally with. 10 ml of 10% sodium chloride solution dichloromethane layer evaporated to get residue. 1.0 gm HPLC purity 90% yield 93%
4) 1 gms (2.06 millimole) of I was suspended 20 ml Dichloromethane along with 2 gm (26.28 millimole) of Propane1,3 diol 25-35° C. and charged 2 gm (18.84 millimole) of trimethyl orthoformate. the reaction mass stirred for 12-18 hours till the TLC analysis indicates the absence of starting material. Add triethylamine to adjust pH alkaline followed by 10 ml water addition and finally with. 10 ml of 10% sodium chloride solution dichloromethane layer evaporated to get residue. 0.9 gm
5) 1 gms (2.06 millimole) of I was suspended 20 ml Toluene along with 1 gm (16.1 millimole) of Ethylene Glycol 25-35° C. and charged 0.5 gm (4.71 millimole) of trimethyl orthoformate. the reaction mass stirred for 6-18 hours till the TLC analysis indicates the absence of starting material. add triethylamine to adjust pH alkaline followed by 10 ml water addition and finally with. 10 ml of 10% sodium chloride solution dichloromethane layer evaporated to get residue. 1.06 gm yield 95%, purified by methanol 0.8 gm purity 95%.

### Step 3: Synthesis of 3-Ketal Cholestenone

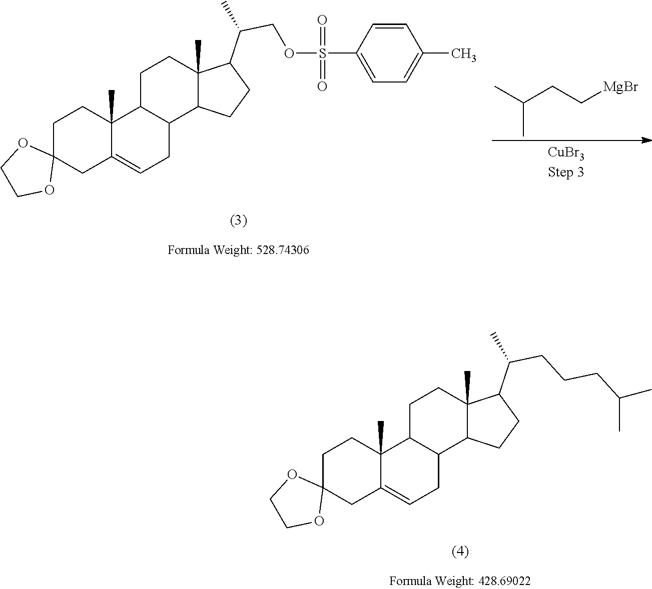

1) 100 gms (4166 millimole) of Magnesium turnings was suspended in 50 ml of Dry Tetrahydrofuran at 40-50° C. under nitrogen atmosphere. 1.0 gm (6.62 millimole) of isopentyl bromide was added at 40-50° C. drop wise to initiate the reaction with help of 1,2 dibromoethane once the vigorous effervescence started slowly addition 200 gm (1324 millimole) of Isopentyl bromide and reaction mass was stir for 60 minutes. Reaction mass is cooled to 0-10° C. catalyst cuprous bromide dimethyl sulphide 1.0 gm (4.86 millimole) and final compound step 2 100 gm (189 millimole) was added simultaneously stir till the TLC indicates the completion of reaction. The reaction mass was cooled to 10° C., Quenched reaction mass to 10% ammonium chloride solution 500 ml then extracted in ethyl acetate 500 ml, to the extracted ethyl acetate The entire ethyl acetate layer was then evaporated under vacuum and the residue (65.0 gm) Yield 90% HPLC purity 85%.
2) 50 gms (2083 millimole) of Magnesium turnings was suspended in 500 ml of Dry Tetrahydrofuran at 50-60° C. under nitrogen atmosphere. 1 gm (6.62 millimole) of isopentyl bromide was added at 40-50° C. drop wise to initiate the reaction with help of 1,2 dibromoethane once the vigorous effervescence started slowly addition 100 gm (662 millimole) of Isopentyl bromide and reaction mass was stir for 60 minutes. Reaction mass is cooled to 0-10° C. catalyst cuprous bromide dimethyl sulphide 1.0 gm (4.86 millimole) and final compound step 2 100 gm (189 millimole) was added simultaneously stir till the TLC indicates the completion of reaction. The reaction mass was cooled to 10° C., Quenched reaction mass to 10% ammonium chloride solution 500 ml then extracted in 500 ml ethyl acetate, to the extracted ethyl acetate The entire ethyl acetate layer was then evaporated under vacuum and the residue (80 gm) HPLC purity 85% yield purified in acetone: 25 gm.
3) 5.0 gms (208.4 millimole) of Magnesium turnings was suspended in 50 ml of Methyl-tertbutyl ether at 40-50° C. under nitrogen atmosphere. 1 gm (6.62 millimole) of isopentyl bromide was added at 40-50° C. drop wise to initiate the reaction with help of 1,2 dibromoethane once the vigorous effervescence started slowly addition 15 gm (97.37 millimole) of Isopentyl bromide and reaction mass was stir for 60 minutes. Reaction mass is cooled to 0-10° C. catalyst cuprous bromide dimethyl sulphide 0.1 gm (0.486 millimole) and final compound step 2 10 gm (18.9 millimole) dissolved in Methyl-tertButyl ether 50 ml was added simultaneously stir till the TLC indicates the completion of reaction. The reaction mass was cooled to 10° C., Quenched reaction mass to ammonium chloride solution then extracted in Methyl-tertbutyl Ether, The entire organic layer was then evaporated under vacuum and the residue 3.2 gm crystalized using alcohol to yield 1.5 gm HPLC purity 91%.
4) 5.0 gms (208.4 millimole) of Magnesium turnings was suspended in 50 ml of Dry Diethyl Ether at 40-50° C. under nitrogen atmosphere. 1 gm (6.62 millimole) of isopentyl bromide was added at 28-32°° C. drop wise to initiate the reaction with help of 1,2 dibromoethane once the vigorous effervescence started slowly addition 15 gm (97.37 millimole) of Isopentyl bromide and reaction mass was stir for 60 minutes. Reaction mass is cooled to 0-10° C. catalyst cuprous bromide dimethyl sulphide 0.1 gm (0.486 millimole) and final compound step 2 10 gm (18.9 millimole) dissolved in diethyl ether 50 ml was added simultaneously stir till the TLC indicates the completion of reaction. The reaction mass was cooled to 10° C., Quenched reaction mass to ammonium chloride solution then extracted in Diethyl Ether, The organic layer layer was then evaporated under vacuum and the residue 3.0 gm crystalized using alcohol to yield 2.0 gm HPLC purity 92% yield 45%.
5) 5.0 gms (208 millimole) of Magnesium turnings was suspended in 50 ml of 2-Methyl Tetrahydrofuran at 55-60° C. under nitrogen atmosphere. 1 gm (6.62 millimole) of isopentyl bromide was added at 55-60° C. drop wise to initiate the reaction with help of 1,2 dibromoethane once the vigorous effervescence started slowly addition 15 gm (97.37 millimole) of Isopentyl bromide and reaction mass was stir for 60 min. Reaction mass is cooled to 0-10° C. catalyst cuprous bromide dimethyl sulphide 0.1 gm (0.486 millimole) and final compound step 2 10 gm (18.9 millimole) dissolved in diethyl ether 50 ml was added simultaneously stir till the TLC indicates the completion of reaction. The reaction mass was cooled to 10° C., Quenched reaction mass to ammonium chloride solution then extracted in Diethyl Ether, The organic layer was then evaporated under vacuum and the residue 3.0 gm crystalized using alcohol to yield 1.0 gm HPLC purity 87% yield 45%.
   Step 4: Synthesis of Cholest-4-ene-3-none.

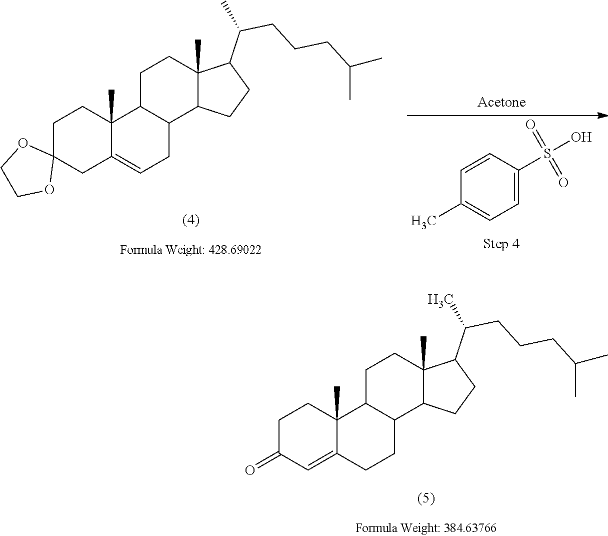

1) To final residue of step 3 compound 4, 100 gm (224 millimole), Added Ethyl acetate 500 ml, added 25 gm (131.4 millimole) para toluene sulphonic acid monohydrate in 100 ml water stirred the mass for 3-8 hrs till tlc complies washed with 200 ml of water. The entire ethyl acetate layer was then evaporated under vacuum and residue obtained 80 gm HPLC purity 87% yield 89%.
2) To final residue of step 3 compound 4, 5.0 gm (11.2 millimole), Added Ethyl acetate 50 ml, 2.5 ml HCL stirred the mass for 3-8 hrs till tlc complies washed with 20 ml of water. The entire ethyl acetate layer was then evaporated under vacuum and residue obtained 3.9 gm, crystalized with alcohol gives 1.5 gm HPLC purity 95% yield.
3) To organic layer of step 3 compound 4, 50 gm (112 millimole), Added Ethyl acetate 100 ml, 25 ml HCL stirred the mass for 3 hrs till tlc complies washed with 20 ml of water. The entire ethyl acetate layer was then evaporated under vacuum and residue obtained 41 gm, HPLC purity 86% yield 86% crystallised with methanol 30 gm.
   Step 5: Synthesis of 3-Acetoxy-Cholest-3,5-diene.

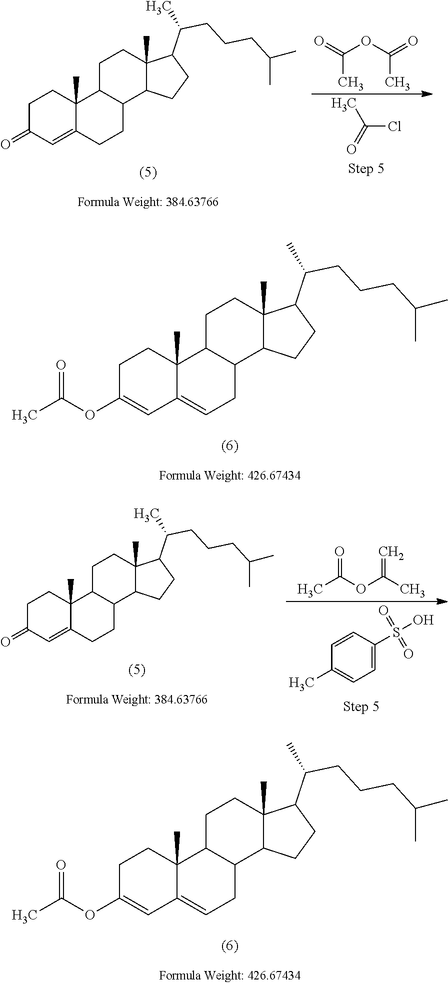

### Examples:

1) 100 gms (259 millimole) of step 4 residue was suspended in 300 gm (2938 millimole) of Acetic Anhydride and 100 gm (1274 millimole) of Acetyl chloride at 25-35° C. and the reaction mass stirred for 6-8 hours at 105-110° C. till the TLC analysis indicates the absence of starting, cool reaction mass to ambient temperature quinch reaction mass in Ice cold water solution followed by 500 ml water and finally extracted with 500 ml of Dichloromethane, washed organic layer with 250 ml of 8% sodium bicarbonate solution dichloromethane layer evaporated to get residue 90 gm HPLC purity 82% yield 81%, purified with acetone 50 gm HPLC purity 91% Yield 45%.
2) 50 gms (129.5 millimole) of step 4 residue was suspended in 100 gm (998.8 millimole) Isopropenyl acetate and 0.1 ml of sulphuric acid at 25-35° C. and the reaction mass stirred for 6-8 hours at 100° C. till the TLC analysis indicates the absence of starting, cool reaction mass to ambient temperature quinch reaction mass in Ice cold water solution followed by extraction with Dichloromethane 500 ml and finally wash with. 50 ml of 10% sodium bicarbonate solution dichloromethane layer evaporated to get residue. 52.0 gm yield 94% crystalised with methanol 30 gm HPLC purity 92% yield 54%.
3) 1.0 gms (2.5 millimole) of step 4 residue was suspended in 5 gm (9.9 millimole) Isopropenyl acetate and 0.1 gm of para Toluene sulphonic acid at 25-35° C. and the reaction mass stirred for 6-8 hours at 100° C. till the TLC analysis indicates the absence of starting, cool reaction mass to ambient temperature quinch reaction mass in Ice cold water solution followed by extraction with Dichloromethane and finally wash with. 50 ml of 10% sodium bicarbonate solution dichloromethane layer evaporated to get residue. 1.0 gm crystalised with methanol 0.5 gm HPlc purity 90% yield 45%.
4) 1.0 gms (2.5 millimole) of step 4 residue was suspended in 1 gm (9.98 millimole) Isopropenyl acetate and 0.1 ml of sulphuric acid, 10 ml of Toluene at 25-35° C. and the reaction mass stirred for 6-8 hours at 100° C. till the TLC analysis indicates the absence of starting, cool reaction mass to ambient temperature quinch reaction mass in Ice cold water solution followed by extraction with Dichloromethane and finally wash with. 50 ml of 10% sodium bicarbonate solution dichloromethane layer evaporated to get residue. 0.9 gm. Yield 81% HPLC purity 87% crystallized with methanol 0.6 gm.

### Step 6: Synthesis of Cholesterol as Major Product and Epicholesterol as Side Product.

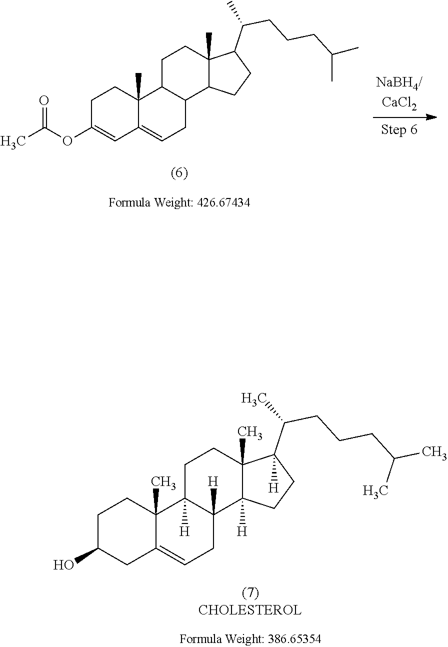

### Examples:

1) To 60 gm (540.6 millimole) of calcium chloride charged absolute Ethanol 600 ml stir cool the mass to 5-10 Degrees then slowly added sodium borohydride 50 gm (1321 millimole) allow to stir for 30 min at 5-10 degree, 100 gm of compound 6 (226 millimole) dissolve in MDC 100 ml slowly added, at 5-10° C. then stir 25-32 degree temperature for 12-24 hrs reaction compliance checked on TLC. Quenched Reaction mass in ice cold water 1.0 lit extracted with 1.0 lit Dichloromethane wash with 2 N HCL 250 ml solution to yield crude product 85 gm, HPLC purity 85% epicholesterol 10%

### Purification:

Dissolve above crude in 20% toluene in methanol solution 250 ml, Heated to 65°° C. maintain 30 minutes, allow to cool to 25-30° C. gradually, chill to 10-15° C., filter it wash by methanol 20 ml

Dry the solid product under vacuum at 50° C.

Dry weight 65 gm

### HPLC Purity 96%

### Epicholesterol 2%

2) To 60 gm (540.6 millimole) of calcium chloride charged absolute Ethanol 600 ml stir cool the mass to 5-10 Degrees then slowly added sodium borohydride 50 gm (1321 millimole) allow to stir for 30 min at 20-30 degree, 100 gm of compound 6 (226 millimole) dissolve in MDC 100 ml slowly added, at 25-30° C. for 12-24 hrs reaction compliance checked on TLC. Quenched Reaction mass in ice cold water 1.0 lit, extracted with 1000 ml DCM wash with 2 N HCL solution 250 ml to yield crude product 80 gm, HPLC purity 70% epicholesterol 20%

Purification by column chromatography:

Dissolve crude in Toluene load on silica column packed with 60-200 mesh size in Toluene.

Elute with 5% Acetone in Toluene, separation can be analysed by TLC to separate the Epicholesterol 8 gm

Final main fraction crystalized in methanol 200 ml Dry weight 50 gm, HPLC purity 96.5%,
3) To 6 gm (54.06 millimole) of calcium chloride charged in methanol 100 ml stir cool the mass to 5-10 Degrees then slowly added sodium borohydride 5.0 gm (132.1 millimole) allow to stir for 30 min at 40-45 degree, 10 gm of compound 6 (22.6 millimole) dissolve in Toluene 10 ml slowly added, at 40-45° C. then stir for 12-18 hrs reaction compliance checked on TLC. Quenched Reaction mass in ice cold water extracted with 50 ml Toluene wash with 2 N HCL solution 20 ml to yield crude product yield 3.0 gm HPLC purity 65% epicholesterol 25%.

Final product achieved by chromatography as in example 3 yield 1.2 gm.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

We claim:

1. A short, cost effective process for the preparation of cholesterol from bisnoralcohol comprising;
i. Tosylating Bisnoralcohol to obtain bisnoralcohol tosylate (2);
ii. Converting the compound (2) to 3-Ketal BisnoralcoholTosylate (3) with ethylene glycol in presence of alkyl ester of orthoformic acid;
iii. Treating the compound (3) with Grignard reagent to obtain 3-Ketal Cholestenone (4);
iv. Deprotecting compound (4) to give Cholest-4-ene-3-none (5) in presence of acid and water;
V. Acetylating compound (5) to give 3-Acetoxy-Cholest-3,5-diene (6); and
vi. Reducing the compound (6) to obtain cholesterol by in-situ generation of calcium borohydride.

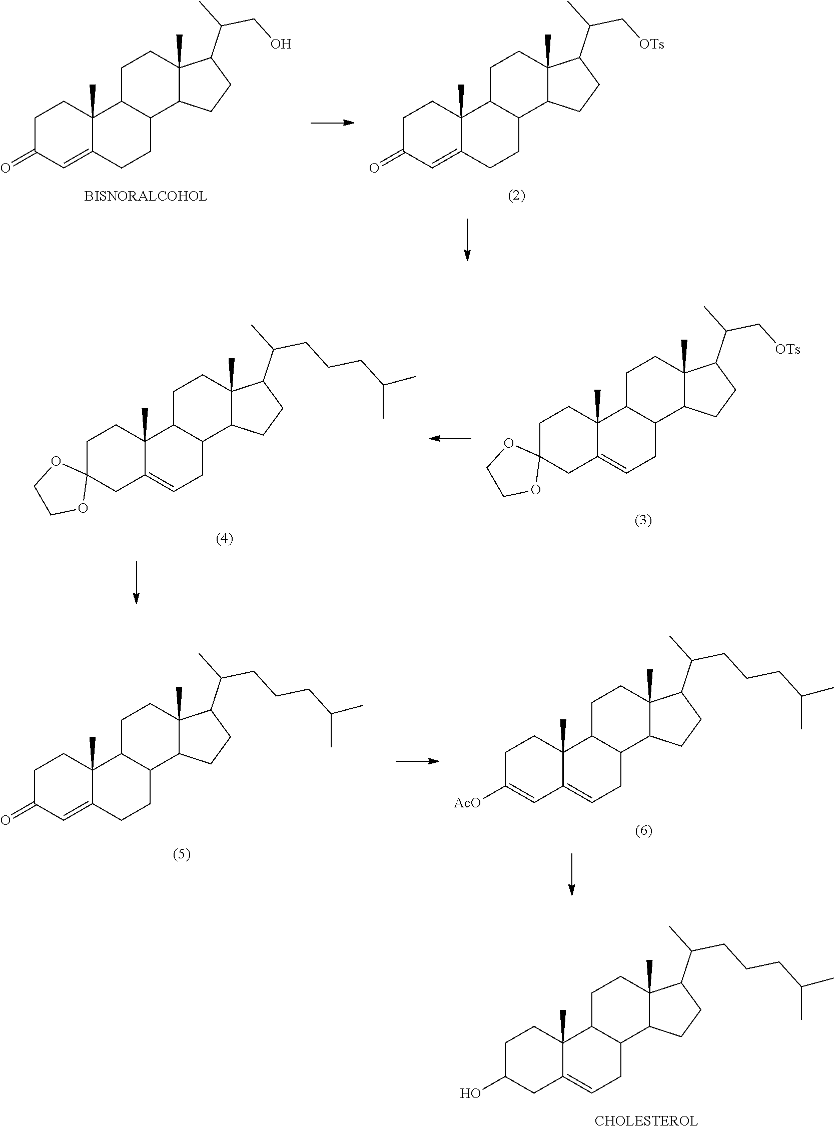

2. The process as claimed in claim 1, wherein process step (i) is carried out in presence of base selected from pyridine, triethyl amine, imidazole or Diisopropyl ethyl amine and the solvent selected from halogenated hydrocarbons, toluene, esters and the like at a temperature ranging between 25-45° C.

3. The process as claimed in claim 1, wherein process step (ii) is carried out using Para toluene sulphonic acid monohydrate in presence of Propane1,3 diol; the alkyl ester of orthoformic acid is selected from trimethyl orthoformate, triethyl orthoformate and the like at a temperature ranging between 25-120° C.

4. The process as claimed in claim 1, wherein process step (iii) is carried out in presence of cuprous bromide, dimethyl sulphide in the solvent selected from THE, diethyl ether or 3-THF.

5. The process as claimed in claim 1, wherein the acid for process step (iv) is selected from organic or inorganic acid.

6. The process as claimed in claim 1, wherein the process step (v) is carried out in presence of acid catalyst, the acetylating agent is selected from acetic anhydride, Isopropenyl acetate and the like at a temperature ranging between 90-120° C.

7. The process as claimed in claim 1, wherein for the process step (vi) the calcium borohydride is generated in situ by reacting alkali metal borohydrides with anhydrous calcium chloride in presence of solvent alone or mixtures thereof; the temperature for step (vi) ranging between 0-25° C.

8. The process as claimed in any one of the preceding claims wherein the solvent for the process is selected from lower alcohols, halogenated hydrocarbons, aliphatic or aromatic hydrocarbons, ethers, diazoles, esters, ketones, nitriles and the like alone or mixtures thereof.

9. The process as claimed in any one of the preceding claims wherein the base is selected from organic or inorganic bases.
